(19) United States
(12) Patent Application Publication
(10) Pub. No.: US 2005/0131457 A1
(43) Pub. Date: Jun. 16, 2005

(54) Variable stiffness shaft

(75) Inventors: Peter Douglas, Milford, NJ (US); Denis LaBombard, Georgetown, MA (US); Gary R. Whipple, Attleboro, MA (US); Stephen Evans, Westford, MA (US)

Correspondence Address: SCULLY SCOTT MURPHY & PRESSER, PC, 400 GARDEN CITY PLAZA, SUITE 300, GARDEN CITY, NY, 11530, US

(73) Assignee: Ethicon, Inc., Somerville, NJ (US)

(21) Appl. No.: 10/736,199

(22) Filed: Dec. 15, 2003

(51) Int. Cl. A61B 17/42

(52) U.S. Cl. 606/205

(57) ABSTRACT

A flexible malleable shaft is made up of a plurality of prismatic shaft elements adjacent one another. A recess is formed in a proximal end of each shaft element, the recess defined along a transverse axis. A protrusion is formed in a distal end of each shaft element, the protrusion defined along a transverse axis. The transverse axes are oriented to one another such that adjacent like shaft elements are aligned with one another when a protrusion of one shaft element is aligned with a recess in an adjacent shaft element. A tension element secured to a distal end of the malleable shaft is in communication with a proximal end of the malleable shaft via an axial through hole. Additionally, a variable stiffness malleable shaft can accommodate the differential lengths of tension elements when applying force to transition the shaft.

### BACKGROUND OF THE INVENTION

1. Field of Invention

The present invention relates to the field of variable stiffness devices and surgical instrumentation. More specifically, it relates to a variable stiffness shaft having means to stiffen the shaft and means to activate a surgical tool carried at a distal end of the shaft independently from one another.

2. Description of Related Art

Variable stiffness devices are used in primarily two situations during a surgical procedures. The first involves the accurate positioning of a surgical device, such as a retractor or stabilizer. A flexible shaft overcomes the difficulty of manipulating a rigid shaft. Once the device is in place, the shaft may be made more rigid, in order to allow the position of the device to be accurately held.

A second situation involves the positioning of multiple surgical devices at the surgical site, thereby congesting the working view or area for the surgeon. This problem is particularly acute when using less invasive and minimally invasive surgical techniques, which are becoming more frequently used for their benefits to the patient. Using a variable stiffness shaft in this circumstance, the surgeon can place or manipulate the device while the shaft is rigid, then transition the shaft to a flexible state, and move the shaft out of the working view or area, thereby improving access and/or visualization.

There are a number of known devices utilizing variable stiffness shafts. Known methods for accomplishing a variable stiffness shaft include cable tension, mechanically telescoping sheaths, and one-dimensional flexibility. These devices are sub-optimal in part because of the large diameter needed to obtain the required stiffness.

Mechanical telescoping devices have a generally flexible shaft that is made stiff by a rigid telescoping sheath extended over it. Once in place, the sheath is retracted, and the flexible shaft may be moved away from the surgical field. At least one drawback of these devices is that the sheath is difficult to retract in vivo.

Cable tension devices suffer from the problem that they will typically manipulate the operation of the surgical tool carried at the distal end of the flexible shaft in the process of stiffening the shaft.

### BRIEF SUMMARY OF THE INVENTION

Therefore, in order to overcome these and other deficiencies in the prior art, provided is a flexible malleable shaft comprising a plurality of generally prismatic shaft elements adjacent one another, each having a first longitudinal axis, and a plurality of axial through holes. A recess is formed in a proximal end of the shaft element, the recess defined along a second axis transverse to the first longitudinal axis and a protrusion is formed in a distal end of the shaft element, the protrusion defined along a third axis transverse to the longitudinal axis. The second and third axes are oriented relative to one another such that the respective axial through holes of adjacent like shaft elements are aligned with one another when a protrusion of one shaft element is aligned with a recess in an adjacent like shaft element. A tension element secured to a distal end of the malleable shaft is in communication with a proximal end of the malleable shaft via an axial through hole.

In another embodiment, a variable stiffness malleable shaft comprises a plurality of tension elements connected to a distal end of the malleable shaft, an actuator for applying force to the plurality of tension elements, a compensation element mounted to articulate about a point in space, and a connector linking the plate to the actuator.

Alternately, a variable stiffness malleable shaft has a first pair of tension elements, each connected between a distal end of the malleable shaft and the other tension element of the pair. A fulcrum has a distal side and a proximal side, with the joined proximal ends of the tension element passing over a proximal side of the fulcrum. An actuator is linked via a connector to the fulcrum, and applies force to the plurality of tension elements. The fulcrum may be a ball, and may have one or more channels to accommodate one or more pairs of tension elements over its proximal side.

### BRIEF DESCRIPTION OF THE DRAWINGS

The foregoing features, benefits, and advantages of the present invention will be made apparent with reference to the following descriptions and figures, wherein like reference numerals refer to like elements across the several views.

FIG. 1 illustrates a surgical instrument according to a first embodiment of the present invention;

FIG. 2A illustrates a shaft element according to the first embodiment;

FIG. 2B illustrates an alternate embodiment of a shaft element according to the present invention;

FIG. 3 illustrates a portion of the assembled malleable shaft section according to the first embodiment;

FIG. 4 illustrates a portion of the assembled malleable shaft according to a second embodiment;

FIG. 5 illustrates an embodiment of the present invention including a malleable shaft section that changes diameter along its length; and

FIG. 6 illustrates a transitional shaft element according to the embodiment of FIG. 5.

FIG. 7 illustrates a further aspect of the present invention for accommodating differential lengths due to the orientation of the malleable shaft.

FIG. 8 illustrates an alternate embodiment for accommodating differential lengths due to the orientation of the malleable shaft.

### DETAILED DESCRIPTION OF THE INVENTION

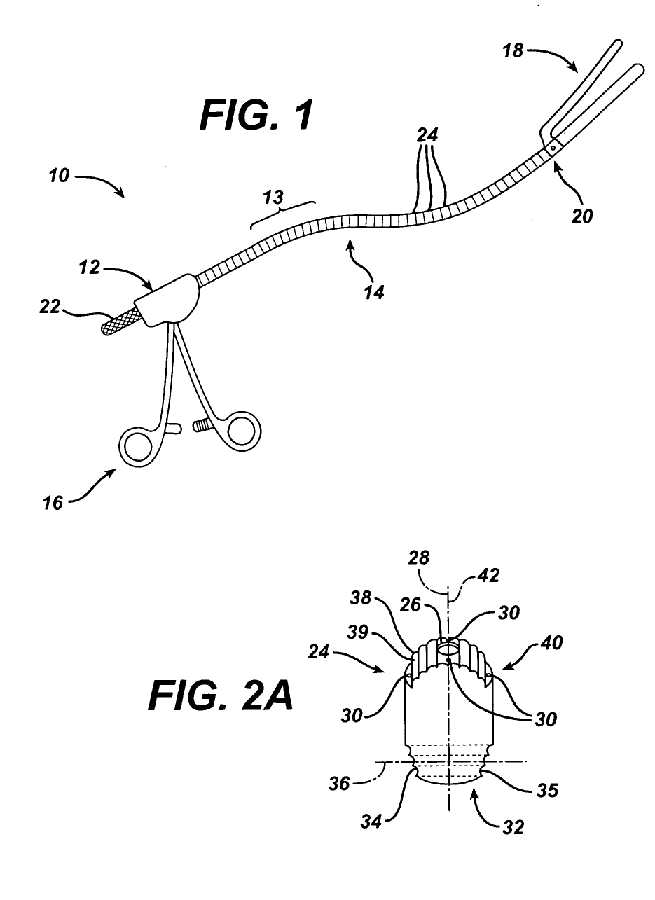

Referring now to FIG. 1, shown is a surgical instrument, generally 10, according to a first embodiment of the present invention. The surgical instrument comprises a proximal base section 12, and a malleable shaft section 14. The base section 12 is adapted for the surgeon to manipulate by hand. It includes actuating levers 16 for actuating a remote apparatus 18 carried on a distal end 20 of the malleable shaft section 14. In this case, the remote apparatus is a clamp. Other remote apparatus contemplated include, but are not limited to, surgical retractors or stabilizers, which may or may not include remote actuation, ligation, ablation, and endoscopy tools. Neither is the present invention limited to only one remote apparatus.

In alternate embodiments, the proximal base section may be additionally or alternately adapted for securement to additional surgical hardware, including, but not limited to, a surgical retractor or other apparatus. Base section 12 will also include an actuator 22 to transition the malleable shaft portion between flexible and rigid states. A lead screw, among other means, is known in the art to transition a malleable shaft between flexible and rigid states. A preferred embodiment of a malleable shaft actuator is disclosed in U.S. patent application Ser. No. 10/609,726, filed 30 Jun. 2003, which is hereby incorporated by reference in its entirety for all purposes.

Malleable shaft section 14 may be integrally formed with the base section 12, or may be adapted to be removable from and/or interchangeable with one or more embodiments of base section 12. Malleable shaft section 14 includes a shaft 16, comprised of a plurality of shaft elements 24.

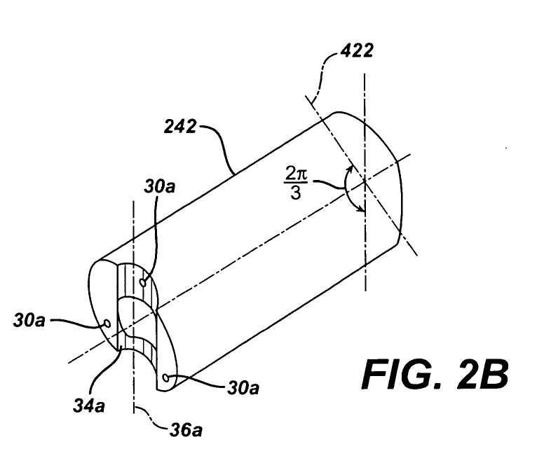

Referring now to FIG. 2A, a shaft element 24 according to a first embodiment of the present invention is shown. Shaft element 24 is generally prismatic in shape, in this case generally cylindrical. A generally prismatic shape will be understood to be that substantially encompassed by a volume extending between two substantially parallel geometric faces. Shaft element 24 has a central through hole 26 generally aligned with a first longitudinal axis 28 of the shaft element 24. Preferably, through hole 26 is substantially parallel with the first longitudinal axis 28, and even more preferably centered on it. A plurality of distributed axial through holes 30, in this embodiment four (4), are distributed about the longitudinal axis 28.

The shaft element 24 has a proximal end 32 having a recess 34 formed therein. Recess 32 is defined along a second transverse axis 36. A protrusion 38 is formed at a distal end 40 of the shaft element 24. Protrusion 38 is defined along a third transverse axis 42, which extends out of the plane of FIG. 2A. Second transverse axis 36 and third transverse axis 42 are oriented relative to one another such that the central axial through hole 26 and distributed axial through holes 30 of a first shaft element 24 are aligned with the central axial through hole 26 and distributed axial through holes 30 of an adjacent shaft element 24 when the transverse axis 42 defining the protrusion 38 of the first shaft element 24 is aligned with the transverse axis 36 defining the recess of the adjacent shaft element 24. With respect to the relationship of adjacent through holes, aligned will be taken to mean that a distal or proximal opening of one axial through hole, whether distributed 30 or central 26, is positioned to coincide with the proximal or distal opening, respectively, of the corresponding through holes in the adjacent shaft element 24, thereby forming an open passage through both shaft elements.

In the exemplary embodiment, transverse axes 36 and 42 are oriented at 90 degrees to one another, and four (4) distributed axial through holes 30 are spaced at or about 90 degree intervals. In an alternate embodiment, shown in FIG. 2B, a shaft element 24a has three (3) distributed axial through holes 30a, and transverse axes 36a and 42a are oriented at or about 120 degrees to one another. Other possible combinations of transverse axis orientation and distributed axial through hole placement will therefore be apparent to those skilled in the art in light of the foregoing disclosure.

Referring again to FIG. 2A, either or both of protrusion 38 and recess 34 may additionally be formed with a friction-enhancing geometry, for example micro-teeth 39 and 35, respectively, or other random or pseudo-random generalized surface roughness. Alternately or additionally, the surfaces may be formed with at least a coating of a high-friction material such as a polyurethane or silastic.

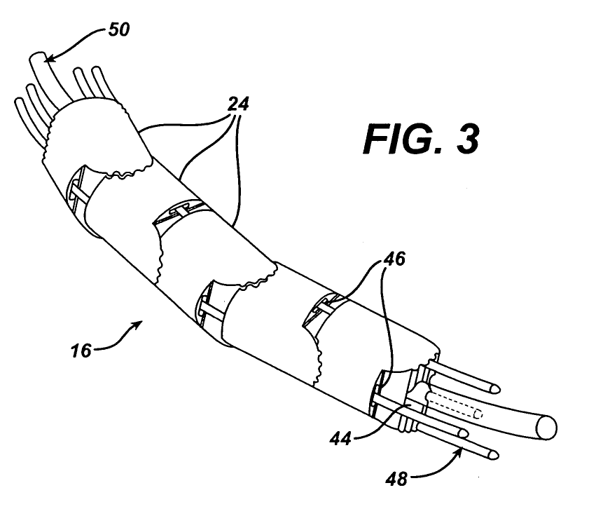

Referring now to FIG. 3, a portion of the assembled malleable shaft 16 is shown in greater detail. A plurality of shaft elements 24 are arranged adjacent one another and oriented such that the protrusion 38 of one shaft element 24 is aligned with the recess 36 of another shaft element 24. Accordingly, the central through holes 26 of adjacent shaft elements 24 form an open central passage 44. Similarly, the distributed axial through holes 30 of adjacent shaft elements 24 form open distributed passages 46. At least one of the distributed passages 46, and more preferably each distributed passage 46, provides clearance for tension elements 48 to run through the plurality of shaft elements 24. The central passage 44 provides clearance for a device actuation cable 50 to run through the plurality of shaft elements 24, where the malleable shaft section 14 is provided with a distal apparatus 18 whose utility is enhanced by remote actuation, as in the case of the clamp jaws shown in the embodiment of FIG. 1.

In operation, each tension element 48 that is provided will be secured to a distal end 20 of the malleable shaft section 14. Each will pass through the length of the shaft section 14, via one of distributed passages 46. Further, each will be operatively connected to an actuator 22 in the proximal base section 12. Actuator 22 is operative to apply force to each tension element 48, and thereby transition the malleable shaft section 14 from a flexible to a rigid state.

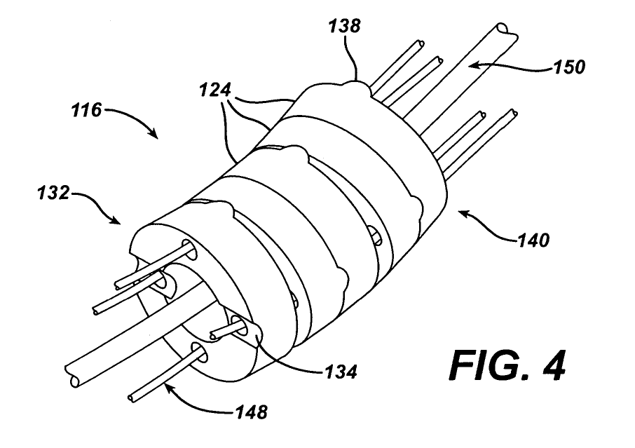

Referring now to FIG. 4, a portion of the assembled shaft 116 according to a second embodiment of the present invention is shown in greater detail. The similarities between the first and second embodiments will be apparent. Malleable shaft section 114 comprises a plurality of shaft elements 124. Shaft elements 124 are shorter in the longitudinal dimension than their counterparts of the first embodiment. Additionally, the size of the recess 134 in a proximal end 132 of shaft element 124 and the size of the protrusion 138 in a distal end 140 of each shaft element 124 will be seen as significantly smaller in both height and width. This has the effect of limiting the angular freedom of each shaft element 124. However, this is compensated for by the fact that the shaft elements are significantly shorter in the longitudinal dimension. The result is that, overall, the flexibility of the shaft in its flexible state is not compromised.

Additionally, the smaller angular displacements impose correspondingly smaller side loads than larger angular displacements, and a shaft under smaller side loads is generally more rigid for a given diameter. Those skilled in the art will appreciate that the choice of shaft element length and maximum angular displacement can be customized to individual applications without departing from the scope of the present invention. Further, the recess 134 and/or the protrusion 138 can be provided with one or more type of friction-enhancing treatment including, but not limited too, micro teeth, random or pseudo-random generalized surface roughness, or a coating layer or more of high-friction material.

It is desirable that the diameter of the malleable shaft section 14 be as thin as possible to improve visualization and access at the surgical site. However, a minimum diameter is approached where the shaft can no longer hold its position while under the loads applied along its length or specifically at the distal end 20. Additionally, the shaft must accommodate within it the distributed passages 46 for tension elements 48, and optionally a central passage 44. It is further apparent that these loads are greater at a proximal portion of the malleable shaft section 14. However, rather than dimension the entire length of the shaft to a diameter necessary to accommodate the loads at the proximal end, it is contemplated that the diameter may change in some manner over the length of the shaft.

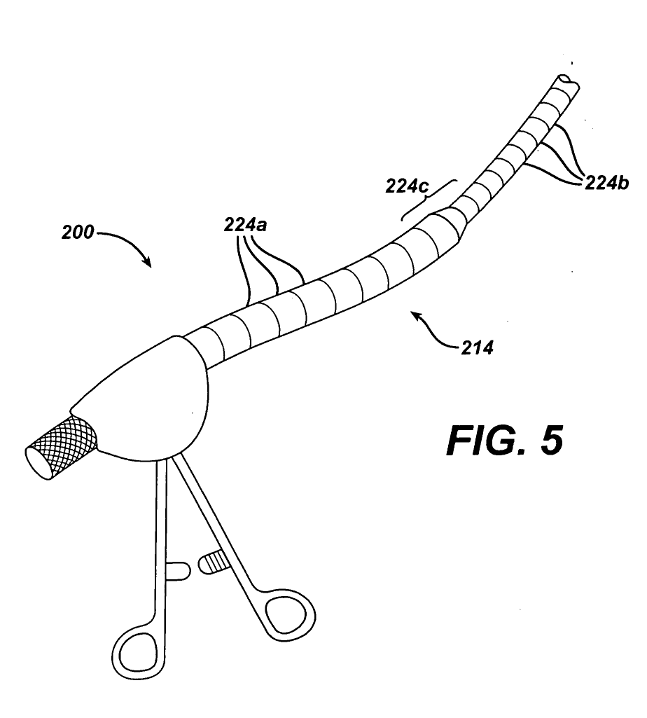

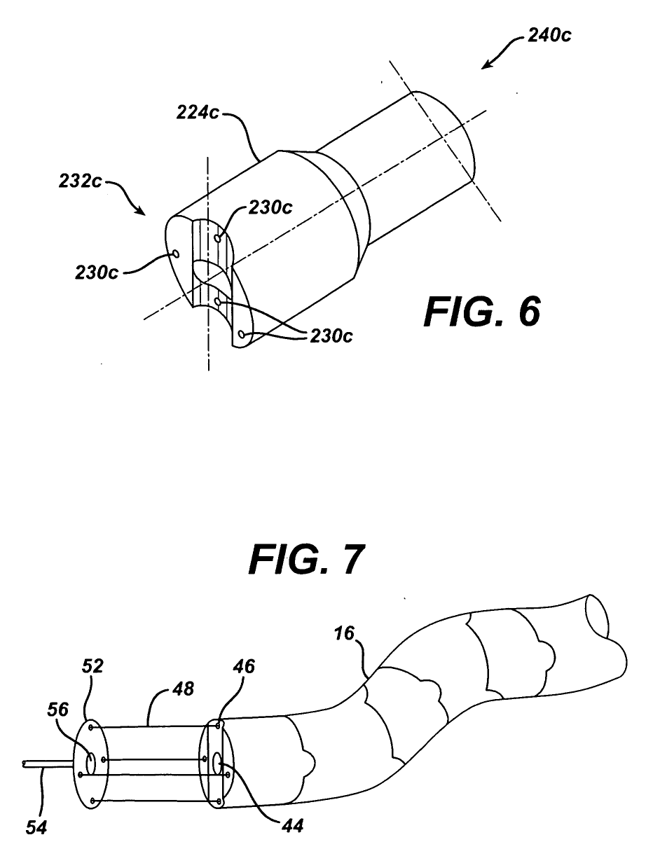

Referring now to FIG. 5, another embodiment 200 of the present invention including a malleable shaft section 214 that changes diameter along its length is shown. In this embodiment, the shaft is comprised of a proximal first plurality of first shaft elements 224a, and a distal second plurality of second shaft elements 224b. These first shaft elements 224a and second shaft elements 224b may be generally similar to each other, but for their size. Further, a transitional shaft element 224c is provided at the interface between the first shaft elements 224a and second shaft elements 224b. Transitional shaft element 224c, shown in greater detail in FIG. 6, will have a proximal end 232c which is generally similar to a proximal end of a first shaft element 224a, and a distal end 240c which is generally similar to a distal end of a second shaft element 224b. Distributed axial through holes 230c will adjust position accordingly with the change in size, shape, and/or diameter to effect the transition.

Alternately, each shaft element may be formed to progressively decrease in size along the length of the malleable shaft and/or include some size variation along its own length. Such size variation along the length, for example a smooth or discontinuous taper, should remain construed within the scope of the generally prismatic description as applied to shaft elements.

Referring now to FIG. 7, as the shape of malleable shaft section 16 is manipulated, the lengths of each distributed passage 46 may differ slightly, because each axial through hole 30 is located off of the longitudinal axis 28 of each shaft element 24. Therefore, location of the proximal ends of each tension element 48 will similarly differ slightly, presuming each tension element is of equal length, because the precise orientation of the shaft generally cannot be predetermined. However, when applying force to the tension elements, it is desirable to apply the force uniformly. Generally, force is applied to the tension elements 48 by displacing the proximal ends proximally. If these ends are fixed in or near the base section 12, then the tension applied may not be uniform, due to the passage length variations. Therefore, it would be desirable to have a means for accommodating the differential lengths when applying force.

FIG. 7 illustrates a further aspect of the present invention. Each tension element 48 is secured to a compensation element, for example swash plate 52. Swash plate 52 is attached to tension rod 54 at a ball joint 56. Tension rod 54 need not be rigid, and a cable or filament may be substituted to connect swash plate 52 with actuator 22. Through the ball joint 56, with support (not shown) by either or both of base section 12 and malleable shaft section 14, the center of the swash plate 52 is generally fixed in space relative to the shaft 16, preferably along the longitudinal axis 28 of a first shaft element 24. The swash plate 52 is free to articulate around any axis. Swash plate 52 may optionally include a clearance area within itself for passage of the actuation cable 50 or the like. In an alternate embodiment, the compensation element may not be a plate at all, but may be replaced by any structure having arms to connect with tension elements 48 around central ball joint 56.

In this embodiment, to transition the malleable shaft 16 from a flexible state to rigid state, the tension rod 54 is displaced proximally under the influence of actuator 22. The freedom of motion of the swash plate 52 allows each tension element to be displaced uniformly. Optionally, when the malleable shaft section 14 is separable from the base section 12, the swash plate will be incorporated into the malleable shaft section 14, with the tension rod 54 extending proximally to interface with the actuator 22 in the base section 12.

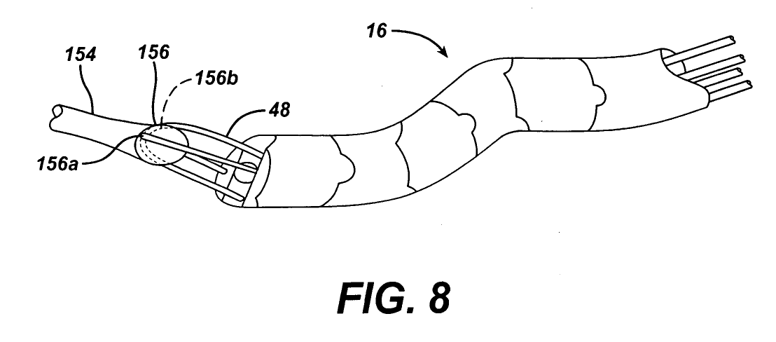

Referring now to FIG. 8, an alternate means for accommodating the differential lengths of passages 46 is shown. In the embodiment of FIG. 8, diametrically opposed tension elements 48 are connected at their proximal ends. In this exemplary embodiment, a ball element 156 has one or more channels, 156a, 156b, formed substantially aligned with an equator or great circle of the ball 156 on its proximal side. Preferably, one channel 156b is set deeper into the ball 156 than another channel 156a. Each channel 156a, 156b is also aligned with the diameter connecting its respective pair of tension elements 48.

The each pair of tension elements is then set into a respectively aligned channel 156a, 156b. As the length of passages 44 change, the tension elements ride over the proximal side of the ball 156 in the channels 156a, 156b, shifting length from one side to the other accordingly. Because the channels 156a, 156b are set to differing depths, crossing tension elements 48 do not interfere with one another. To transition the malleable shaft 16 between flexible to rigid states, ball 156 is displaced proximally via connecting rod 154.

Though the exemplary embodiment in FIG. 8 includes a ball 156, suitable substitutes need not be a ball per se, but merely structure to provide a fulcrum or pivot around which the connected tension elements 48 may reverse direction relative to the opposing tension element 48. The channels 156a, 156b, are optional, and if provided need not overlap.

It is further contemplated that in place of the arrangements disclosed, other pre-tensioning means may be provided for each tension element 48, including, but not limited to a spring in any form known in the art. Further, the transition of the malleable shaft 16 from flexible to rigid states would include transitioning the tension load from the pre-tensioning means through the action of the actuator 22.

The present invention has been described herein with respect to certain preferred embodiments. These embodiments are meant to be illustrative, and not limiting, of the scope of the present invention, which is defined by the appended claims.

1. A variable stiffness malleable shaft comprising:
a plurality of generally prismatic shaft elements adjacent one another, each having:
a first longitudinal axis;
a plurality of axial through holes;
a recess formed in a proximal end of the shaft element, the recess defined along a second axis transverse to the first longitudinal axis; and
a protrusion formed in a distal end of the shaft element, the protrusion defined along a third axis transverse to the longitudinal axis, wherein the second and third axes are oriented relative to one another such that the respective axial through holes of adjacent like shaft elements are aligned with one another when a protrusion of one shaft element is aligned with a recess in an adjacent like shaft element; and
at least one tension element secured to a distal end of the malleable shaft and in communication with a proximal end of the malleable shaft via an axial through hole.

2. The variable stiffness malleable shaft according to claim 1 further comprising at least one remote apparatus located at a distal end of said shaft.

3. The variable stiffness malleable shaft according to claim 2, wherein said at least one remote apparatus is articulated via a central passage formed in said flexible malleable shaft.

4. The variable stiffness malleable shaft according to claim 2, wherein said at least one remote apparatus is selected from the group comprised of a clamp, a scissors, a retractor, a stabilizer, a ligator, an ablator, and an endoscope.

5. The variable stiffness malleable shaft according to claim 1, wherein said shaft elements further comprise a central axial through hole, positioned such that the central axial through holes of adjacent shaft elements are aligned with one another when a protrusion of one shaft element is aligned with a recess in an adjacent like shaft element.

6. The variable stiffness malleable shaft according to claim 1, wherein, in at least one of said shaft elements, at least one of said recess and said protrusion comprises a friction enhancement means.

7. The variable stiffness malleable shaft according to claim 6, wherein said friction enhancement means comprises at least one of a friction enhancing material, and a friction enhancing geometry.

8. The variable stiffness malleable shaft according to claim 1 further comprising a base section adapted for securement to additional surgical hardware.

9. The variable stiffness malleable shaft according to claim 1, wherein said second and third transverse axes are oriented at 90 degrees to one another.

10. The variable stiffness malleable shaft according to claim 9, wherein said plurality of axial through holes comprises four axial through holes distributed approximately 90 degrees from one another about said first longitudinal axis.

11. The variable stiffness malleable shaft according to claim 1, wherein said second and third transverse axes are oriented approximately 120 degrees to one another.

12. The variable stiffness malleable shaft according to claim 11, wherein said plurality of axial through holes comprises three axial through holes distributed approximately 120 degrees from one another about said first longitudinal axis.

13. The variable stiffness malleable shaft according to claim 1, further comprising a plate mounted to articulate about a point in space, and connected to said tension element.

14. An element for use in a variable stiffness malleable shaft comprising, the element comprising:
a generally prismatic body defining a first longitudinal axis;
a plurality of axial through holes;
a recess formed in a proximal end of the element, the recess defined along a second axis transverse to the first longitudinal axis; and
a protrusion formed in a distal end of the element, the protrusion defined along a third axis transverse to the longitudinal axis, wherein the second and third axes are oriented relative to one another such that the axial through holes of adjacent elements are aligned with one another when a protrusion of one shaft element is aligned with a recess in an adjacent shaft element.

15. A variable stiffness malleable shaft comprising:
a plurality of tension elements, each being connected at its distal end to a distal end of the malleable shaft;
a compensation element mounted to articulate about a point in space, wherein each tension element is connected at its proximal end to the compensation element;
an actuator for applying force to the plurality of tension elements; and
a connector linking the compensation element to the actuator.

16. The variable stiffness malleable shaft according to claim 15, wherein the plate is mounted on a ball joint, said ball joint located at one end of said connector.

17. The variable stiffness malleable shaft according to claim 15, wherein said plate comprises a clearance passage.

18. A variable stiffness malleable shaft comprising:
a first pair of tension elements, each tension element connected at its distal end to a distal end of the malleable shaft and at its proximal end to the other tension element of the first pair;
a fulcrum having a distal side and a proximal side, wherein the joined proximal ends of the tension element pass over a proximal side of the fulcrum;
an actuator for applying force to the plurality of tension elements; and
a connector linking the fulcrum to the actuator.

19. The variable stiffness malleable shaft according to claim 18, wherein the fulcrum is generally spherical.

20. The variable stiffness malleable shaft according to claim 19, wherein the fulcrum further comprises a channel in its proximal side for accommodating the pair of tension elements.

21. The variable stiffness malleable shaft according to claim 20, wherein the channel is substantially aligned with a great circle of the spherical fulcrum.

22. The variable stiffness malleable shaft according to claim 18, wherein the fulcrum further comprises a channel in its proximal side for accommodating the pair of tension elements.

23. The variable stiffness malleable shaft according to claim 18, further comprising a second pair of tension elements, each tension element connected at its distal end to a distal end of the malleable shaft and at its proximal end to the other tension element of the second pair.

24. The variable stiffness malleable shaft according to claim 23, wherein the fulcrum further comprises a first channel and a second channel in its proximal side for accommodating the first pair and the second pair of tension elements.

25. The variable stiffness malleable shaft according to claim 24, wherein said second channel is formed deeper in the fulcrum than said first channel.

26. The variable stiffness malleable shaft according to claim 24, wherein said first channel and said second channel cross on a proximal side of said fulcrum.
